(19) United States
(12) Patent Application Publication
(10) Pub. No.: US 2023/0407098 A1
(43) Pub. Date: Dec. 21, 2023

(54) METHOD FOR EVALUATING CYSTINE UPTAKE ABILITY OF CELL, KIT FOR EVALUATING CYSTINE UPTAKE ABILITY OF CELL, METHOD FOR DETERMINING SELENOCYSTEINE, AND KIT FOR DETERMINING SELENOCYSTEINE

(71) Applicant: DOJINDO LABORATORIES, Kamimashiki-gun Kumamoto (JP)

(72) Inventors: Munetaka ISHIYAMA, Kamimashiki-gun Kumamoto (JP); Yuya OHUCHI, Kamimashiki-gun Kumamoto (JP); Takashi SHIMOMURA, Kamimashiki-gun Kumamoto (JP); Norio HIRAKAWA, Kamimashiki-gun Kumamoto (JP)

(21) Appl. No.: 18/460,688

(22) Filed: Sep. 5, 2023

Related U.S. Application Data
(63) Continuation of application No. PCT/JP2022/006280, filed on Feb. 17, 2022.

(30) Foreign Application Priority Data
Mar. 5, 2021 (JP) 2021-034870

(51) Int. Cl. C09B 62/36; G01N 33/68; C09K 11/07; C09B 11/08; G01N 21/64

(52) U.S. Cl. CPC C09B 62/365; G01N 33/68; C09K 11/07; C09B 11/08; G01N 21/6428; C09K 2211/1018; G01N 2021/6439

(57) ABSTRACT

Disclosed are a method for evaluating the cystine uptake ability of cells having the steps of contacting cells with selenocystine to allow the cells to take up selenocystine, washing away selenocystine not taken up by the cells, and crushing the cells and determining selenocystine contained in the cytoplasm and a method for determining selenocysteine having a step of contacting selenocysteine with a fluorescent dye that specifically reacts with selenocysteine to change in one or both of fluorescence wavelength and fluorescence intensity, or reacting selenocysteine with a fluorescent dye under a condition that the fluorescent dye specifically reacts with selenocysteine to change in one or both of fluorescence wavelength and fluorescence intensity, followed by measuring the fluorescence intensity for determining selenocystine. A kit for evaluating the cystine uptake ability of cells, containing a reducing agent that reduces selenocystine to selenocysteine, a fluorescent dye represented by the following general formula (I), and a buffer solution at a pH of 5.5 to 6.5 is disclosed.

### CROSS-REFERENCE TO RELATED APPLICATIONS

This application is a continuation of PCT application No. PCT/JP2022/006280 filed on Feb. 17, 2022, which is based on and claims priority to Japanese Patent Applications No. 2021-034870 filed on Mar. 5, 2021, the disclosures of which are incorporated herein by reference.

### TECHNICAL FIELD

The present invention relates to a novel method for evaluating the cystine uptake ability of cells, a kit for evaluating the cystine uptake ability of cells, a method for determining selenocysteine, and a kit for determining selenocysteine.

### BACKGROUND ART

Amino acid transport through mammalian cell membranes has been frequently investigated since around the 1950s, and some amino acid transport systems have been identified based on the substrate specificity, sodium dependence, and the like as indices (see Non-Patent Literature 1). Substances having roles in such amino acid transport systems are membrane proteins generically named amino acid transporters, and almost all the amino acid transporter genes have been subjected to molecular cloning. The function of amino acid transporters is fundamentally the transport of amino acids inside and outside cells, but some amino acid transporters are related to various physiological functions. Cystine transporter is also one of them, and while a cystine transport system named the X_{c}_{^{−}} system has been identified, and it is revealed that cystine participates in resistance to oxidative stress, the maintenance of the extracellular redox balance, immunocyte activation, and the like with the discovery of the importance of the cystine in cultured cell lines, cystine also attracts attention as a target molecule for treating cancer such as brain tumors (see Non-Patent Literature 2).

The evaluation of the ability to take cystine into cells through the cystine transporter is important for evaluating the activity of the cystine transporter or the inhibitory activity of specific compounds on cystine transporter. Examples of the method for evaluating the ability to take cystine into cells proposed in the past include a method using a fluorescence-labeled cystine derivative or a radioactive isotope-labeled cystine (see Patent Literature 1), a method using a stable isotope-labeled cystine (see Non-Patent Literature 3), and a method for determining glutamic acid released when cystine transporter incorporates cystine (see Non-Patent Literature 4).

### CITATION LIST

### Patent Literature

Patent Literature 1
Japanese Patent No. 5709841

### Non-Patent Literature

Non-Patent Literature 1
“Role of amino acid transport and countertransport in nutrition and metabolism” H. N. Christensen: Physiological Reviews, Vol. 70, p. 43-77 (1990) Non-Patent Literature 2

“Shisuchin/Gurutaminsan Toransupota (Xc-Kei)-Saibo keno Shisuchin Torikomi wo Kaishita Sankasutoresubogyokino to Aratana Tenkai-(Cystine/glutamate Transporter (Xc-system)-Defense Function against Oxidative Stress through Uptake of Cystine into Cells and New Development)” Hideyo Sato, kagakutoseibutsu, Vol. 50, No. 5, p. 316-318 (2012) Non-Patent Literature 3

“Novel mouse model for evaluating in vivo efficacy of xCT inhibitor” Yoshioka, R.; Fujieda, Y.; Suzuki, Y.; Kanno, O.; Nagahira, A.; Honda, T.; Murakawa, M.; Yukiura, H. J. Pharmacol. Sci. 2019, 140, 242-247. Non-Patent Literature 4

“High cell density increases glioblastoma cell viability under glucose deprivation via degradation of the cystine/glutamate transporter xCT (SLC7A11)” Yamaguchi, I.; Yoshimura S. H.; Katoh, H. J. Biol. Chem. 2020, 295, 6936-6945.

### SUMMARY OF INVENTION

### Technical Problem

However, a problem is that the uptake of a fluorescence-labeled cystine derivative into cells through cystine transporter is affected by the fluorescent label. Although the radioisotope label does not affect the uptake into cells through cystine transporter, a problem is that the use of a radioisotope is regulated, and the operation is complicated. A problem is that the method using a stable isotope label is necessary for an expensive mass spectrometer, and the analysis is very difficult in addition. A problem is that the method by measuring glutamic acid released with the uptake of cystine is indirect and lacking in accuracy.

The present invention has been completed in view of such a situation, and an object of the present invention is to provide a method for evaluating the cystine uptake ability of cells that enables evaluating the cystine uptake ability of cells by an easy method at low cost with high accuracy and a kit for evaluating the cystine uptake ability of cells that can be suitably used for the method. In addition, an object of the present invention is to provide a method for determining selenocysteine and a kit for determining selenocysteine.

### Solution to Problem

A first aspect of the present invention matching the object provides a method for evaluating the cystine uptake ability of cells, having the steps of contacting cells with selenocystine to allow the cells to take up selenocystine, washing away selenocystine not taken up by the cells, and crushing the cells and determining selenocystine contained in cytoplasm to solve the problems mentioned above.

In the step of determining selenocystine contained in the cytoplasm in the method for evaluating the cystine uptake ability of cells according to the first aspect of the present invention, it is preferable that selenocystine is reacted with a reducing agent to generate selenocysteine; selenocysteine is contacted with a fluorescent dye that specifically reacts with selenocysteine to change in one or both of the fluorescence wavelength and the fluorescence intensity, or selenocysteine is reacted with a fluorescent dye under the condition that the fluorescent dye specifically reacts with selenocysteine to change in one or both of the fluorescence wavelength and the fluorescence intensity; and the fluorescence intensity is measured to determine selenocystine.

In the step of determining selenocystine contained in the cytoplasm in the method for evaluating the cystine uptake ability according to the first aspect of the present invention, it is preferable that a fluorescent dye represented by the following general formula (I) is specifically reacted with selenocysteine under the condition of a pH of 5.5 to 6.5,

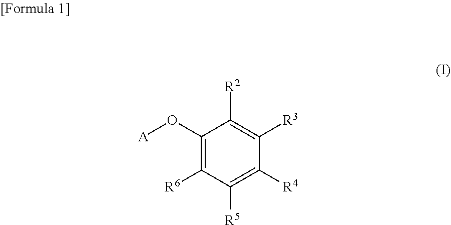

wherein A represents an acryloyl group or a methacryloyl group, R², R³, R⁴, R⁵, and R⁶ are each independently an atom or an atomic group selected from the group consisting of a hydrogen atom, a hydroxyl group, a thiol group, a halogen atom, an amino group, a sulfonamide group, an azido group, and a cyano group; and a linear alkyl group, a branched alkyl group, a cycloalkyl group, an aryl group, and a heteroaryl group in which one or more hydrogen atoms are optionally replaced with other atoms or functional groups and that optionally contain one or more of an amino group, a carbonyl group, an oxygen atom, and a sulfur atom in the carbon skeletons, and among R², R³, R⁴, R⁵, and R⁶, any two adjacent ones share atoms, forming a cyclic fluorescent moiety where the fluorescence wavelength and fluorescence intensity change one or both, due to the conversion of the 0-A group to an OH group.

In the step of determining selenocystine contained in the cytoplasm in the method for evaluating the cystine uptake ability of cells according to the first aspect of the present invention, it is preferable that a fluorescent dye represented by the following formula (1) is specifically reacted with selenocysteine under the condition of a pH of 5.5 to 6.5,

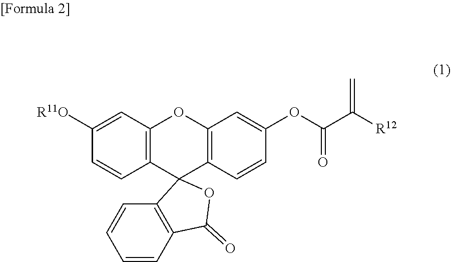

wherein R¹¹ represents a hydrogen atom or a functional group represented by the following formula (2), and R¹² represents a hydrogen atom or a methyl group,

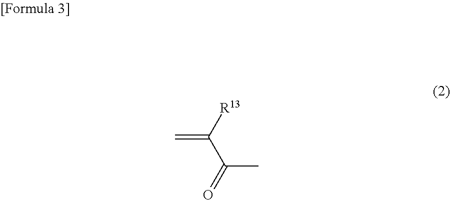

wherein R¹³ represents a hydrogen atom or a methyl group.

In the method for evaluating the cystine uptake ability of cells according to the first aspect of the present invention, it is preferable that the fluorescent dye is represented by the following formula:

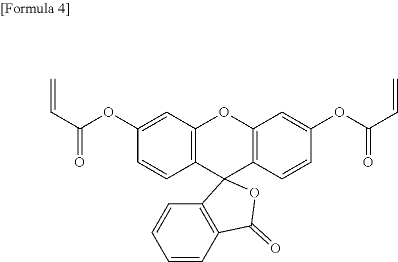 [SMILES: C=CC(=O)Oc1ccc2c(c1)Oc1cc(OC(=O)C=C)ccc1C21OC(=O)c2ccccc21]

In the method for evaluating the cystine uptake ability of cells according to the first aspect of the present invention, the reducing agent may be tris(carboxyethyl)phosphine.

A second aspect of the present invention provides a kit for evaluating the cystine uptake ability of cells, containing a reducing agent that reduces selenocystine to selenocysteine, a fluorescent dye represented by the following general formula (I), and a buffer solution at a pH of 5.5 to 6.5,

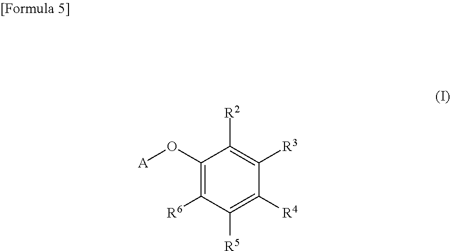

wherein A represents an acryloyl group or a methacryloyl group, R², R³, R⁴, R⁵, and R⁶ are each independently an atom or an atomic group selected from the group consisting of a hydrogen atom, a hydroxyl group, a thiol group, a halogen atom, an amino group, a sulfonamide group, an azido group, and a cyano group; and a linear alkyl group, a branched alkyl group, a cycloalkyl group, an aryl group, and a heteroaryl group in which one or more hydrogen atoms are optionally replaced with other atoms or functional groups and that optionally contain one or more of an amino group, a carbonyl group, an oxygen atom, and a sulfur atom in the carbon skeletons, and among R², R³, R⁴, R⁵, and R⁶, any two adjacent ones share atoms, forming a cyclic fluorescent moiety where the fluorescence wavelength and fluorescence intensity change one or both, due to the conversion of the 0-A group to an OH group, to solve the problems mentioned above.

In the kit for evaluating the cystine uptake ability of cells according to the second aspect of the present invention, it is preferable that the fluorescent dye is represented by the following formula (1):

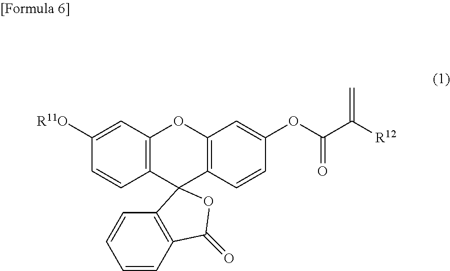

wherein R¹¹ represents a hydrogen atom or a functional group represented by the following formula (2), and R¹² represents a hydrogen atom or a methyl group,

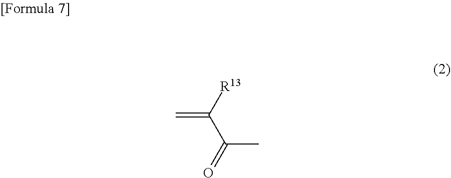

wherein R¹³ represents a hydrogen atom or a methyl group.

In the kit for evaluating the cystine uptake ability of cells according to the second aspect of the present invention, it is preferable that the fluorescent dye is represented by the following formula:

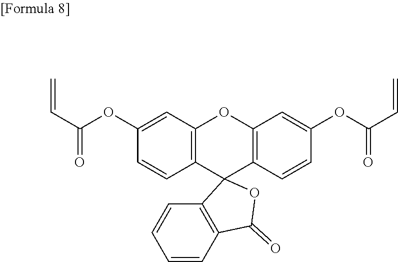 [SMILES: C=CC(=O)Oc1ccc2c(c1)Oc1cc(OC(=O)C=C)ccc1C21OC(=O)c2ccccc21]

In the kit for evaluating the cystine uptake ability of cells according to the second aspect of the present invention, the reducing agent may be tris(carboxyethyl) phosphine.

In the kit for evaluating the cystine uptake ability of cells according to the second aspect of the present invention, the buffer solution may be any of acetate buffer solution, phosphate buffer solution, citrate buffer solution, MES buffer solution, and Bis-Tris buffer solution.

A third aspect of the present invention provides a method for determining selenocysteine having a step of contacting selenocysteine with a fluorescent dye that specifically reacts with selenocysteine to change in one or both of the fluorescence wavelength and the fluorescence intensity, or reacting selenocysteine with a fluorescent dye under the condition that the fluorescent dye specifically reacts with selenocysteine to change in one or both of the fluorescence wavelength and the fluorescence intensity, followed by measuring the fluorescence intensity for determining selenocystine to solve the problems mentioned above.

In the step of determining selenocystine contained in the cytoplasm in the method for determining selenocysteine according to the third aspect of the present invention, it is preferable that the fluorescent dye represented by the following general formula (I) is specifically reacted with selenocysteine under the condition of a pH of 5.5 to 6.5,

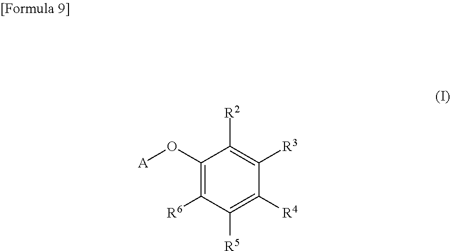

wherein A represents an acryloyl group or a methacryloyl group, R², R³, R⁴, R⁵, and R⁶ are each independently an atom or an atomic group selected from the group consisting of a hydrogen atom, a hydroxyl group, a thiol group, a halogen atom, an amino group, a sulfonamide group, an azido group, and a cyano group; and a linear alkyl group, a branched alkyl group, a cycloalkyl group, an aryl group, and a heteroaryl group in which one or more hydrogen atoms are optionally replaced with other atoms or functional groups and that optionally contain one or more of an amino group, a carbonyl group, an oxygen atom, and a sulfur atom in the carbon skeletons, and among R², R³, R⁴, R⁵, and R⁶, any two adjacent ones share atoms, forming a cyclic fluorescent moiety where the fluorescence wavelength and fluorescence intensity change one or both, due to the conversion of the O-A group to an OH group.

In the step of determining selenocystine contained in the cytoplasm in the method for determining selenocysteine according to the third aspect of the present invention, it is preferable that the fluorescent dye represented by the following formula (1) is specifically reacted with selenocysteine under the condition of a pH of 5.5 to 6.5,

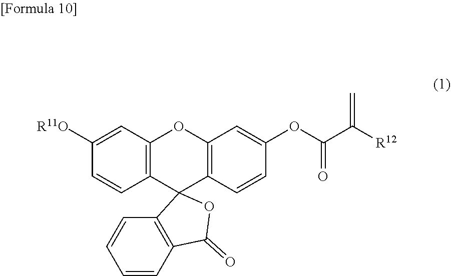

wherein R¹¹ represents a hydrogen atom or a functional group represented by the following formula (2), and R¹² represents a hydrogen atom or a methyl group,

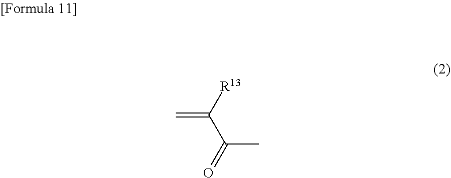

wherein R¹³ represents a hydrogen atom or a methyl group.

In the method for determining selenocysteine according to the third aspect of the present invention, it is preferable that the fluorescent dye is represented by the following formula:

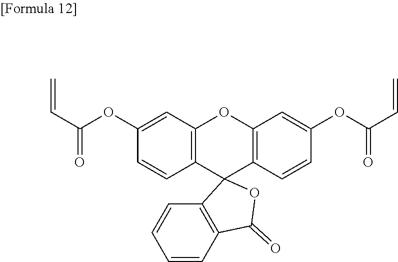 [SMILES: C=CC(=O)Oc1ccc2c(c1)Oc1cc(OC(=O)C=C)ccc1C21OC(=O)c2ccccc21]

In the method for determining selenocysteine according to the third aspect of the present invention, the reducing agent may be tris(carboxyethyl)phosphine.

A fourth aspect of the present invention provides a kit for determining selenocysteine containing the fluorescent dye represented by the following general formula (I) and a buffer solution at a pH of 5.5 to 6.5,

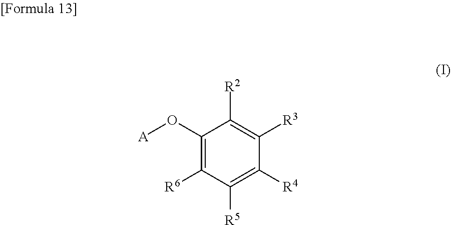

wherein A represents an acryloyl group or a methacryloyl group, R², R³, R⁴, R⁵, and R⁶ are each independently an atom or an atomic group selected from the group consisting of a hydrogen atom, a hydroxyl group, a thiol group, a halogen atom, an amino group, a sulfonamide group, an azido group, and a cyano group; and a linear alkyl group, a branched alkyl group, a cycloalkyl group, an aryl group, and a heteroaryl group in which one or more hydrogen atoms are optionally substituted with other atoms or functional groups and that optionally contain one or more of an amino group, a carbonyl group, an oxygen atom, and a sulfur atom in the carbon skeletons, and among R², R³, R⁴, R⁵, and R⁶, any two adjacent ones share atoms, forming a cyclic fluorescent moiety where the fluorescence wavelength and fluorescence intensity change one or both, due to the conversion of the O-A group to an OH group.

In the kit for determining selenocysteine according to the fourth aspect of the present invention, it is preferable that the fluorescent dye is represented by the following formula (1):

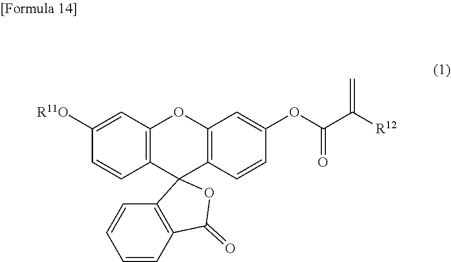

wherein R¹¹ represents a hydrogen atom or a functional group represented by the following formula (2), and R¹² represents a hydrogen atom or a methyl group,

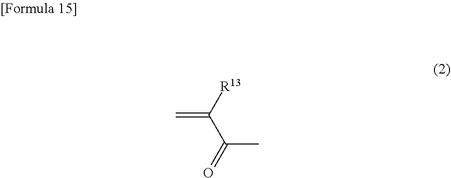

wherein R¹³ represents a hydrogen atom or a methyl group.

In the kit for determining selenocysteine according to the fourth aspect of the present invention, it is preferable that the fluorescent dye is represented by the following formula:

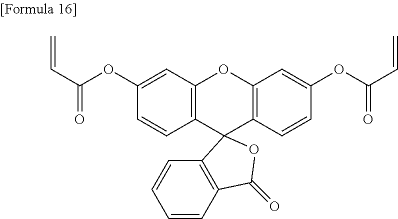 [SMILES: C=CC(=O)Oc1ccc2c(c1)Oc1cc(OC(=O)C=C)ccc1C21OC(=O)c2ccccc21]

In the kit for determining selenocysteine according to the fourth aspect of the present invention, the reducing agent may be tris(carboxyethyl)phosphine.

In the kit for determining selenocysteine according to the fourth aspect of the present invention, the buffer solution may be any of acetate buffer solution, phosphate buffer solution, citrate buffer solution, MES buffer solution, and Bis-Tris buffer solution.

### Advantageous Effects of Invention

The ability of cells to take up selenocystine through cystine transporter is not different from the ability of cells to take cystine through cystine transporter. Since selenocysteine, which is a reduced product of selenocystine, is different from cysteine in chemical properties, selenocysteine can be specifically determined without being disturbed by cysteine, glutathione, or the like present in the cytoplasm. According to the present invention, a method for evaluating the cystine uptake ability of cells that enables evaluating the cystine uptake ability of cells at low cost with high accuracy is provided due to the characteristics mentioned above. According to the present invention, a kit for evaluating the cystine uptake ability of cells that can be suitably used for the method is provided, and a method for determining selenocysteine and a kit for determining selenocysteine are provided in addition.

### BRIEF DESCRIPTION OF DRAWINGS

FIG. 1 is a graph showing the results of Example 1.

FIG. 2 is a graph showing the results of Example 2.

FIG. 3 is a graph showing the results of Example 3.

FIG. 4 is a graph showing the results of Example 3.

### DESCRIPTION OF EMBODIMENTS

A method for evaluating the cystine uptake ability of cells according to the first embodiment of the present invention (hereinafter occasionally abbreviated as a “method for evaluating the cystine uptake ability of cells” or an “evaluation method”) has the steps of contacting cells with selenocystine to allow the cells to take up selenocystine, washing away selenocystine not taken up by the cells, and crushing the cells and determining selenocystine contained in the cytoplasm.

The evaluation object in the evaluation method is any cells in which cystine transporter is expressed, and specific examples thereof include A549 (cells derived from human pulmonary alveoli basal epithelial gland cancer), HeLa (cells derived from human cervical cancer), HepG2 (cells derived from human liver cancer), HL60 (human leukemia cell line), MOLT-4 (cells derived from a human acute lymphoblastic tumor), U-251 (malignant human glioma cell line), U-87 MG (cells derived from human glioblastoma), BxPC-3 (cells derived from human pancreas cancer), THP-1 (cells derived from human acute monocytic leukemia), SK-MEL-30 (cells derived from human skin cancer), EFO-21 (cells derived from human serous cystoma), GAMG (cells derived from a human brain tumor), Karpas-707 (cells derived from human multiple myeloma), OE-19 (cells derived from human esophagus cancer), U266/70 (cells derived from human multiple myeloma), RT4 (cells derived from human bladder epithelial papilloma), and U-2 OS (cells derived from human osteosarcoma).

Cells such as the cells mentioned above as an evaluation object are first contacted with selenocystine to allow the cells to take up selenocystine. Examples of the method for taking selenocystine into cells include a method involving dissolving selenocystine in culture medium or a buffer solution not containing cystine (for example, cystine-free DMEM, HBSS, PBS, or the like), then adding the culture medium or the buffer solution containing selenocystine to cells, and leaving the cells to stand in a CO₂ incubator at 37° C. for around 5 minutes to 1 hour.

Selenocystine not taken up by cells is removed before selenocystine taken up by cells is determined. Examples of the removal method include, but are not particularly limited to, a method for washing the cells with a buffer solution, liquid culture medium, or the like not containing selenocystine.

The cells are then crushed, the cytoplasm is lysed, and selenocystine contained in the cytoplasm is determined to evaluate the selenocystine uptake ability. The method for crushing cells is not particularly limited, and any well-known method can be used. Examples include a method for lysing cells with a water-soluble organic solvent such as an alcohol, DMSO or a buffer solution containing a surfactant.

Selenocystine in the eluted cytoplasm is then determined. Specific examples of the method for determining selenocysteine include a method involving reducing selenocystine to produce selenocysteine; contacting the selenocysteine with a fluorescent dye that specifically reacts with selenocysteine to emit fluorescence or reacting a fluorescent dye with selenocysteine under the condition that the fluorescent dye specifically reacts with selenocysteine to emit fluorescence; and measuring the fluorescence intensity to determine selenocystine.

Specific examples of a preferable reducing agent to be used for reducing selenocystine include tris(2-carboxyethyl) phosphine (TCEP), having many advantages such as having no smell or no toxicity and being excellent in hydrophilicity and stability.

Selenocysteine is then contacted with a fluorescent dye that specifically reacts with selenocysteine to change in one or both of the fluorescence wavelength and the fluorescence intensity even in the presence of cysteine, or selenocysteine is reacted with a fluorescent dye under the condition that the fluorescent dye specifically reacts with selenocysteine to change in one or both of the fluorescence wavelength and the fluorescence intensity even in the presence of cysteine; and the fluorescence intensity is measured to determine selenocystine.

Examples of the preferable dye and the condition of the specific reaction include specifically reacting a fluorescent dye represented by the following general formula (I) with selenocysteine under the condition of a pH of 5.5 to 6.5,

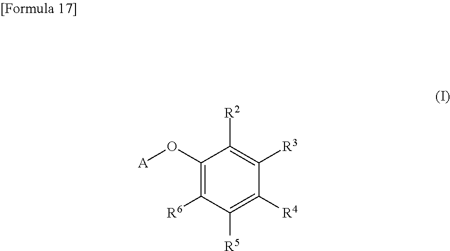

wherein A represents an acryloyl group or a methacryloyl group, R², R³, R⁴, R⁵, and R⁶ are each independently an atom or an atomic group selected from the group consisting of a hydrogen atom, a hydroxyl group, a thiol group, a halogen atom, an amino group, a sulfonamide group, an azido group, and a cyano group; and a linear alkyl group, a branched alkyl group, a cycloalkyl group, an aryl group, and a heteroaryl group in which one or more hydrogen atoms are optionally replaced with other atoms or functional groups and that optionally contain one or more of an amino group, a carbonyl group, an oxygen atom, and a sulfur atom in the carbon skeletons, and among R², R³, R⁴, R⁵, and R⁶, any two adjacent ones share atoms, forming a cyclic fluorescent moiety where the fluorescence wavelength and fluorescence intensity change one or both, due to the conversion of the 0-A group to an OH group.

Specific examples of the fluorescent dye represented by general formula (I) to be specifically reacted with selenocysteine under the condition of a pH of 5.5 to 6.5 include fluorescent dyes represented by the following formulae:

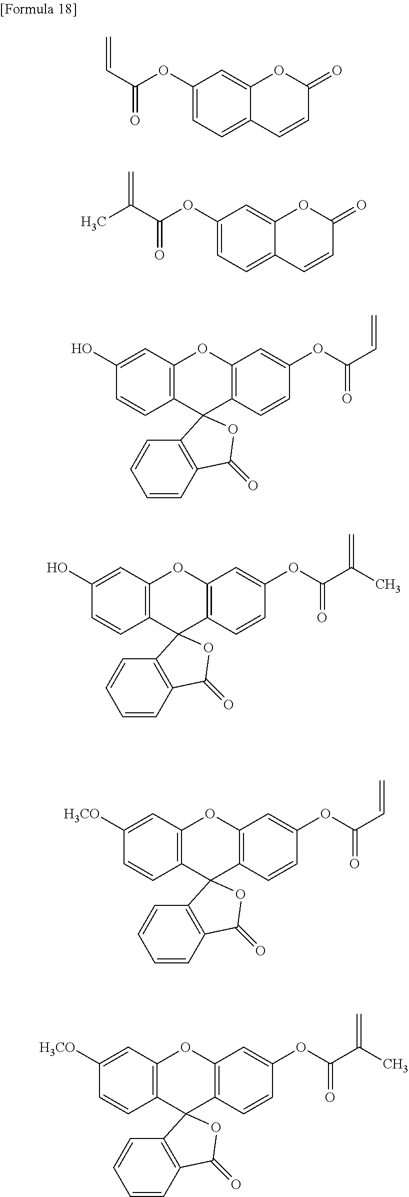

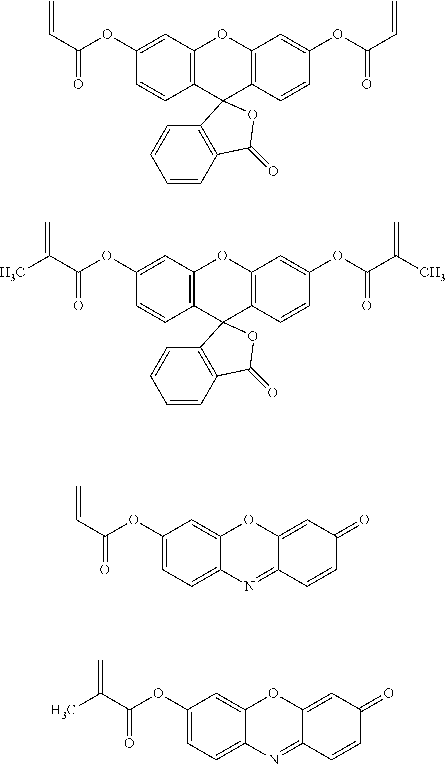

Examples of a particularly preferable fluorescent dye include a substance represented by the following formula (1):

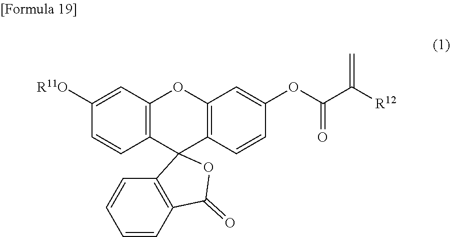

wherein R¹¹ represents a hydrogen atom or a functional group represented by the following formula (2), and R¹² represents a hydrogen atom or a methyl group,

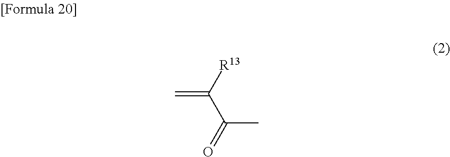

wherein R¹³ represents a hydrogen atom or a methyl group.

In the case of the fluorescent dye represented by the formula mentioned above (1), the specific reactions of fluorescent dye with selenocysteine and the reaction mechanism thereof are represented, for example, by the following formula.

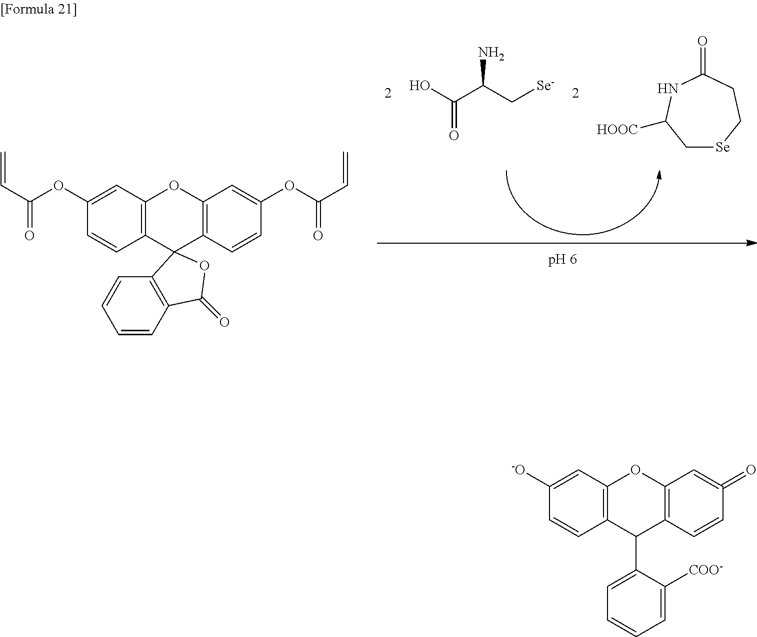

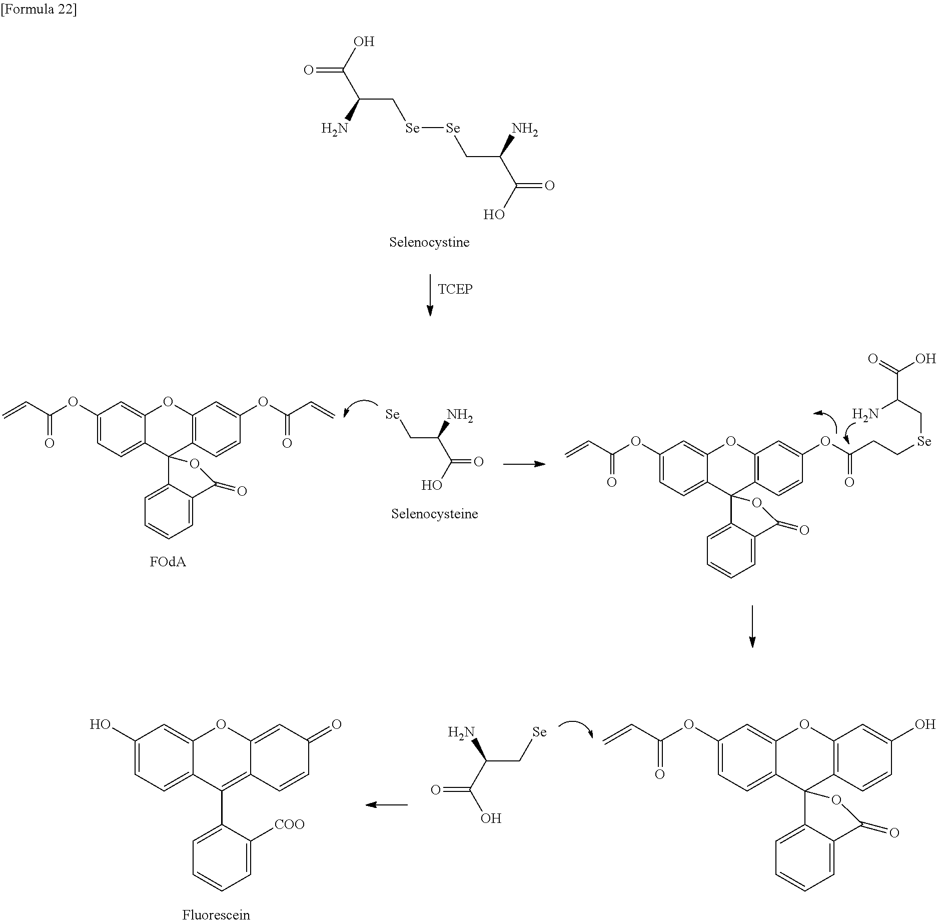

Examples of preferable buffer solution include acetate buffer solution, phosphate buffer solution, citrate buffer solution, MES buffer solution, and Bis-Tris buffer solution.

The kit for evaluating the cystine uptake ability of cells according to the second embodiment of the present invention contains a reducing agent that reduces selenocystine to selenocysteine, a fluorescent dye represented by the following general formula (I), and a buffer solution at a pH of 5.5 to 6.5. Since specific examples of the reducing agent, the fluorescent dye, and the buffer solution are as described in the above-mentioned first embodiment of the present invention, a detailed description thereof is omitted.

The method for determining selenocysteine according to the third embodiment of the present invention has a step of contacting a fluorescent dye that specifically reacts with selenocysteine to change in one or both of the fluorescence wavelength and the fluorescence intensity or reacting a fluorescent dye with selenocysteine under the condition that the fluorescent dye specifically reacts with selenocysteine to change in one or both of the fluorescence wavelength and the fluorescence intensity; and measuring the fluorescence intensity to determine selenocysteine. Since the steps are as described in the above-mentioned first embodiment of the present invention, a detailed description thereof is omitted.

### EXAMPLES

The Examples performed to confirm the function and the effect of the present invention will then be described.

### Example 1: Examination of Reactivity of Fluorescein O, O′-Diacrylate (FOdA) with Selenocystine

Fluorescein O,O′-diacrylate (FOdA, 10 μM) was mixed with TCEP (200 μM) and any of 10 μM selenocystine, 10 μM cystine, and 20 μM glutathione in buffers (100 mM acetate buffers at pHs of 5 and 5.5, 100 mM MES buffers at pHs of 6 and 6.5, and 100 mM phosphate buffer at a pH of 7), and the mixture was incubated at 37° C. for 30 minutes. The fluorescence intensity of each solution was measured with a plate reader (Infinite(R) 200 PRO, excitation wavelength: 485 nm, fluorescence wavelength: 535 nm).

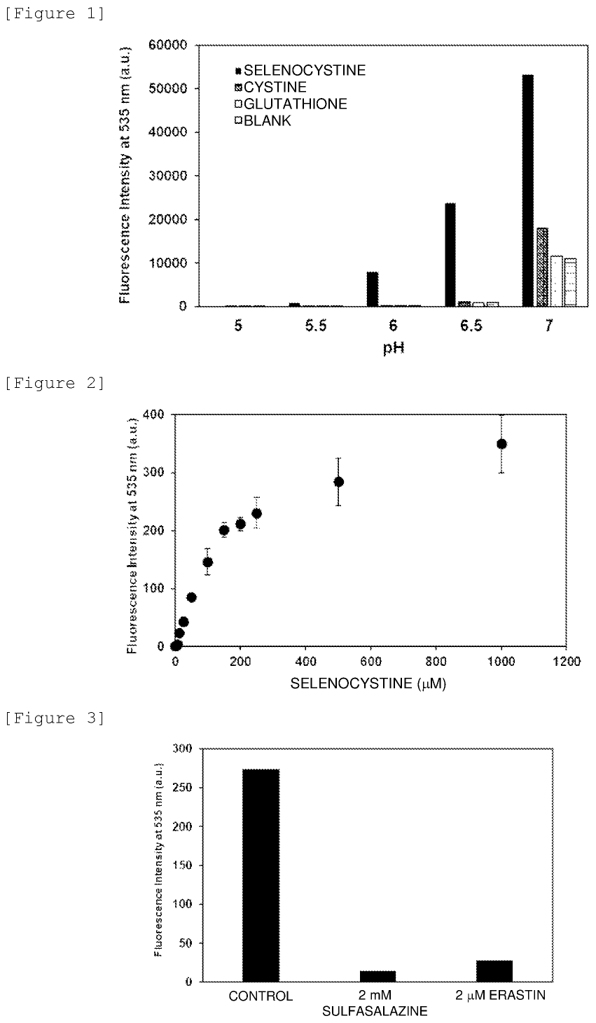

As shown in FIG. 1, it was confirmed that FOdA was hardly reacted with cystine or glutathione in the buffers at pH 6 and 6.5 and selectively reacted with selenocystine to emit fluorescence.

### Example 2: Examination of Ability to Take Selenocystine into Cells

DMEM (cystine-free) was added to HeLa cells, and the cells were incubated in a CO₂ incubator (37° C.) for 5 minutes. After the removal of the supernatant, DMEM (cystine-free) containing selenocystine at concentrations was added, and the cells were incubated in the CO 2 incubator (37° C.) for 30 minutes. After removing the supernatant, the cells were washed with PBS three times and then lysed with methanol. Then, 100 mM MES at a pH of 6 containing 10 μM FOdA and 200 μM TCEP was added, and the mixture was incubated at 37° C. for 30 minutes. The fluorescence intensity of each solution was then measured with a plate reader (Infinite(R) 200 PRO, excitation wavelength: 485 nm, fluorescence wavelength: 535 nm).

As shown in FIG. 2, it was confirmed that the fluorescence intensity increased depending on the concentration of selenocystine to be added to the cells. This result shows that selenocystine is taken up by the cells, and selenocystine taken up by the cells can be subjected to fluorescence measurement by the reaction using FOdA and TCEP.

### Example 3: Examination of Inhibition of Selenocystine Uptake by Cystine Transporter Inhibitor

DMEM (cystine-free) containing a cystine transporter inhibitor (sulfasalazine or erastin) was added to HeLa cells, and the cells were incubated in the CO₂ incubator (37° C.) for 5 minutes. DMEM (cystine-free) containing 200 μM selenocystine was then added, and the cells were incubated in the CO₂ incubator (37° C.) for 30 minutes. After removing the supernatant, the cells were washed with PBS three times and lysed with methanol. Then, 100 mM MES at a pH of 6 containing 10 μM FOdA and 200 μM TCEP was added, and the cells were incubated at 37° C. for 30 minutes. The fluorescence intensity of each solution was then measured with a plate reader (Infinite(R) 200 PRO, excitation wavelength: 485 nm, fluorescence wavelength: 535 nm).

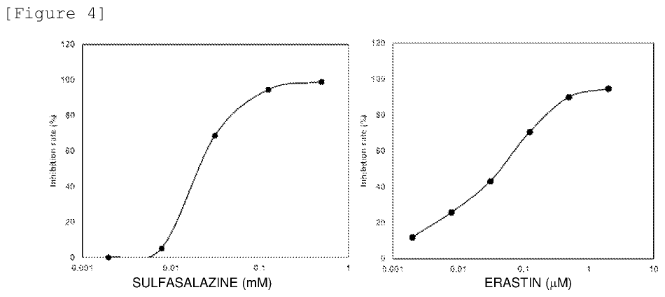

FIG. 3 shows the results. Since the fluorescence intensity significantly decreased in the cells to which the cystine transporter inhibitor was added, it was confirmed that selenocystine was taken through cystine transporter.

As shown in FIG. 4, the cystine transporter inhibitor concentration-dependent inhibitory activity was able to be calculated from this decrease in fluorescence intensity.

The present invention enables various embodiments and modifications without departing from the broad spirit and the scope of the present invention. The above-mentioned embodiments are for describing the present invention, and do not limit the scope of the present invention. That is, the scope of the present invention is shown not by the embodiments but by Claims. Various modifications made within Claims and the meaning of the invention equivalent thereto are considered to be within the scope of the present invention.

The present application is based on Japan Patent Application 2021-34870, filed on Mar. 5, 2021, and includes Description, Claims, Drawings, and Abstract of thereof. The disclosure in the Japan Patent Application mentioned above is incorporated herein in its entirety as a reference.

1. A method for evaluating cystine uptake ability of cells, comprising the steps of:
contacting cells with selenocystine to allow the cells to take up selenocystine; washing away selenocystine not taken up by the cells; and crushing the cells and determining selenocystine contained in cytoplasm.

2. The method for evaluating cystine uptake ability of cells according to claim 1, wherein, in the step of determining selenocystine contained in cytoplasm, selenocystine is reacted with a reducing agent to generate selenocysteine; selenocysteine is contacted with a fluorescent dye that specifically reacts with selenocysteine to change in one or both of fluorescence wavelength and fluorescence intensity, or selenocysteine is reacted with a fluorescent dye under the condition that the fluorescent dye specifically reacts with selenocysteine to change in one or both of fluorescence wavelength and fluorescence intensity; and the fluorescence intensity is measured to determine selenocystine.

3. The method for evaluating cystine uptake ability of cells according to claim 2, wherein, in the step of determining selenocystine contained in the cytoplasm, a fluorescent dye represented by the following general formula (I) is specifically reacted with selenocysteine under a condition of a pH of 5.5 to 6.5, 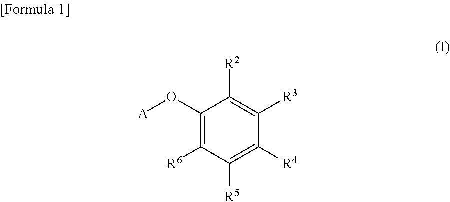
wherein A represents an acryloyl group or a methacryloyl group, R², R³, R⁴, R⁵, and R⁶ are each independently an atom or an atomic group selected from the group consisting of a hydrogen atom, a hydroxyl group, a thiol group, a halogen atom, an amino group, a sulfonamide group, an azido group, and a cyano group; and a linear alkyl group, a branched alkyl group, a cycloalkyl group, an aryl group, and a heteroaryl group in which one or more hydrogen atoms are optionally replaced with other atoms or functional groups and that optionally comprises one or more of an amino group, a carbonyl group, an oxygen atom, and a sulfur atom in carbon skeletons, and among R², R³, R⁴, R⁵, and R⁶, any two adjacent ones share atoms, forming a cyclic fluorescent moiety where the fluorescence wavelength and fluorescence intensity change one or both, due to the conversion of the O-A group to an OH group.

4. The method for evaluating cystine uptake ability of cells according to claim 3, wherein, in the step of determining selenocystine contained in the cytoplasm, a fluorescent dye represented by the following formula (1) is specifically reacted with selenocysteine under the condition of a pH of 5.5 to 6.5, 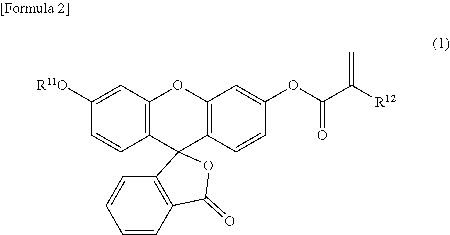
wherein R¹¹ represents a hydrogen atom or a functional group represented by following formula (2), and R¹² represents a hydrogen atom or a methyl group, 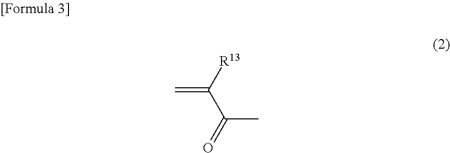
wherein R¹³ represents a hydrogen atom or a methyl group.

5. The method for evaluating cystine uptake ability of cells according to claim 4, wherein the fluorescent dye is represented by the following formula:

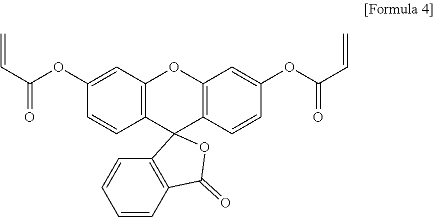 [SMILES: C=CC(=O)Oc1ccc2c(c1)Oc1cc(OC(=O)C=C)ccc1C21OC(=O)c2ccccc21]

6. The method for evaluating cystine uptake ability of cells according to claim 2, wherein the reducing agent is tris(carboxyethyl)phosphine.

7. A kit for evaluating cystine uptake ability of cells, comprising:
a reducing agent that reduces selenocystine to selenocysteine,
a fluorescent dye represented by the following general formula (I), and
a buffer solution at a pH of 5.5 to 6.5, 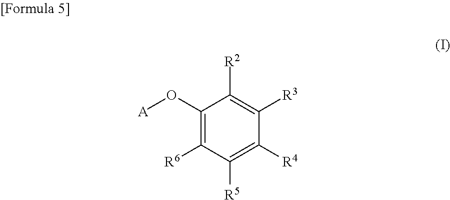
wherein A represents an acryloyl group or a methacryloyl group, R², R³, R⁴, R⁵, and R⁶ are each independently an atom or an atomic group selected from the group consisting of a hydrogen atom, a hydroxyl group, a thiol group, a halogen atom, an amino group, a sulfonamide group, an azido group, and a cyano group; and a linear alkyl group, a branched alkyl group, a cycloalkyl group, an aryl group, and a heteroaryl group in which one or more hydrogen atoms are optionally replaced with other atoms or functional groups and that optionally contain one or more of an amino group, a carbonyl group, an oxygen atom, and a sulfur atom in carbon skeletons, and among R², R³, R⁴, R⁵, and R⁶, any two adjacent ones share atoms, forming a cyclic fluorescent moiety where the fluorescence wavelength and fluorescence intensity change one or both, due to the conversion of the 0-A group to an OH group.

8. The kit for evaluating cystine uptake ability of cells according to claim 7, wherein the fluorescent dye is represented by the following formula (1): 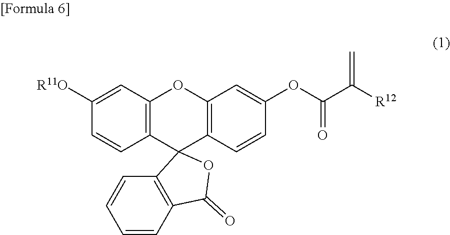
wherein R¹¹ represents a hydrogen atom or a functional group represented by the following formula (2), and R¹² represents a hydrogen atom or a methyl group, 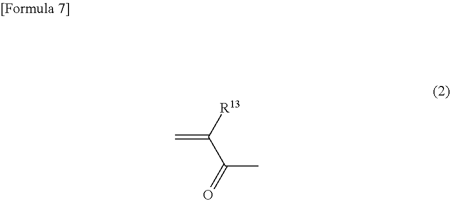
wherein R¹³ represents a hydrogen atom or a methyl group.

9. The kit for evaluating cystine uptake ability of cells according to claim 8, wherein the fluorescent dye is represented by the following formula:

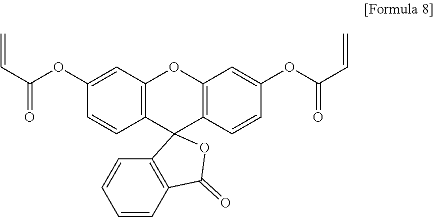 [SMILES: C=CC(=O)Oc1ccc2c(c1)Oc1cc(OC(=O)C=C)ccc1C21OC(=O)c2ccccc21]

10. The kit for evaluating cystine uptake ability of cells according to claim 7, wherein the reducing agent is tris(carboxyethyl)phosphine.

11. The kit for evaluating cystine uptake ability of cells according to claim 7, wherein the buffer solution is any of acetate buffer solution, phosphate buffer solution, citrate buffer solution, MES buffer solution, and Bis-Tris buffer solution.

12. A method for determining selenocysteine, comprising a step of contacting selenocysteine with a fluorescent dye that specifically reacts with selenocysteine to change in one or both of fluorescence wavelength and fluorescence intensity, or reacting selenocysteine with a fluorescent dye under a condition that the fluorescent dye specifically reacts with selenocysteine to change in one or both of fluorescence wavelength and fluorescence intensity, followed by measuring the fluorescence intensity for determining selenocystine.

13. The method for determining selenocysteine according to claim 12, wherein, in the step of determining selenocystine, the fluorescent dye represented by the following general formula (I) is specifically reacted with selenocysteine under a condition of a pH of 5.5 to 6.5, 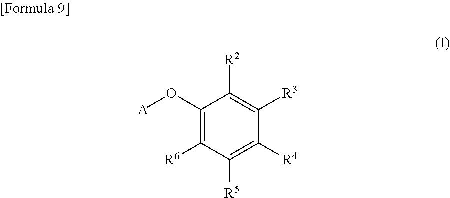
wherein A represents an acryloyl group or a methacryloyl group, R², R³, R⁴, R⁵, and R⁶ are each independently an atom or an atomic group selected from the group consisting of a hydrogen atom, a hydroxyl group, a thiol group, a halogen atom, an amino group, a sulfonamide group, an azido group, and a cyano group; and a linear alkyl group, a branched alkyl group, a cycloalkyl group, an aryl group, and a heteroaryl group in which one or more hydrogen atoms are optionally replaced with other atoms or functional groups and that optionally contain one or more of an amino group, a carbonyl group, an oxygen atom, and a sulfur atom in the carbon skeletons, and among R², R³, R⁴, R⁵, and R⁶, any two adjacent ones share atoms, forming a cyclic fluorescent moiety where the fluorescence wavelength and fluorescence intensity change one or both, due to the conversion of the O-A group to an OH group.

14. The method for determining selenocysteine according to claim 13, wherein, in the step of determining selenocysteine contained in the cytoplasm, the fluorescent dye represented by the following formula (1) is specifically reacted with selenocysteine under a condition of a pH of 5.5 to 6.5, 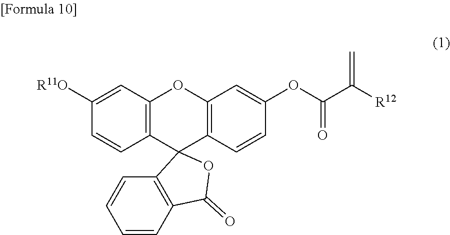
wherein R¹¹ represents a hydrogen atom or a functional group represented by the following formula (2), and R⁴² represents a hydrogen atom or a methyl group, 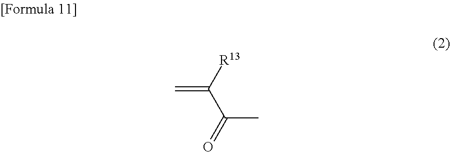
wherein R¹³ represents a hydrogen atom or a methyl group.

15. The method for determining selenocysteine according to claim 14, wherein the fluorescent dye is represented by the following formula:

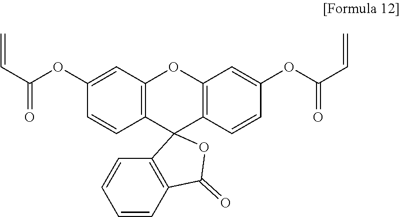 [SMILES: C=CC(=O)Oc1ccc2c(c1)Oc1cc(OC(=O)C=C)ccc1C21OC(=O)c2ccccc21]

16. The method for determining selenocysteine according to claim 12, wherein the reducing agent is tris(carboxyethyl)phosphine.

17. A kit for determining selenocysteine, comprising:
a fluorescent dye represented by the following general formula (I) and
a buffer solution at a pH of 5.5 to 6.5, 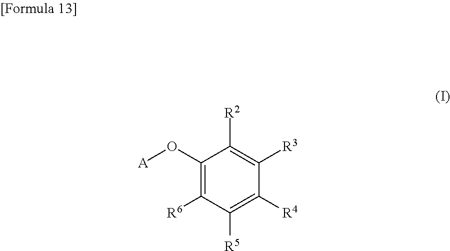
wherein A represents an acryloyl group or a methacryloyl group, R², R³, R⁴, R⁵, and R⁶ are each independently an atom or an atomic group selected from the group consisting of a hydrogen atom, a hydroxyl group, a thiol group, a halogen atom, an amino group, a sulfonamide group, an azido group, and a cyano group; and a linear alkyl group, a branched alkyl group, a cycloalkyl group, an aryl group, and a heteroaryl group in which one or more hydrogen atoms are optionally replaced with other atoms or functional groups and that optionally contain one or more of an amino group, a carbonyl group, an oxygen atom, and a sulfur atom in the carbon skeletons, and among R², R³, R⁴, R⁵, and R⁶, any two adjacent ones share atoms, forming a cyclic fluorescent moiety where the fluorescence wavelength and fluorescence intensity change one or both, due to the conversion of the 0-A group to an OH group.

18. The kit for determining selenocysteine according to claim 17, wherein the fluorescent dye is represented by following formula (1): 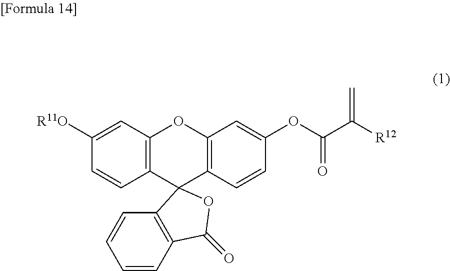
wherein R¹¹ represents a hydrogen atom or a functional group represented by the following formula (2), and R¹² represents a hydrogen atom or a methyl group, 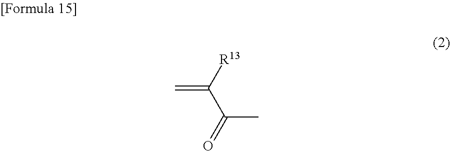
wherein R¹³ represents a hydrogen atom or a methyl group.

19. The kit for determining selenocysteine according to claim 18, wherein the fluorescent dye is represented by the following formula:

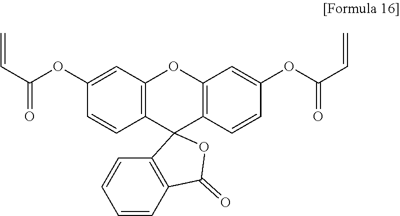 [SMILES: C=CC(=O)Oc1ccc2c(c1)Oc1cc(OC(=O)C=C)ccc1C21OC(=O)c2ccccc21]

20. The kit for determining selenocysteine according to claim 17, wherein the reducing agent is tris(carboxyethyl) phosphine.

21. The kit for determining selenocysteine according to claim 17, wherein the buffer solution is any of acetate buffer solution, phosphate buffer solution, citrate buffer solution, MES buffer solution, and Bis-Tris buffer solution.
